(19) United States
(12) Patent Application Publication
(10) Pub. No.: US 2015/0209365 A1
(43) Pub. Date: Jul. 30, 2015

(54) Methods and Compositions for Treating Luekemia

(71) Applicants: Mhairi Copland, Glasgow (GB); Marion Dorsch, Jamaica Plain, MA (US); David Irvine, Glasgow (GB); Paul W. Manley, Arlesheim (CH); Stefan Peukert, Arlington, MA (US)

(72) Inventors: Mhairi Copland, Glasgow (GB); Marion Dorsch, Jamaica Plain, MA (US); David Irvine, Glasgow (GB); Paul W. Manley, Arlesheim (CH); Stefan Peukert, Arlington, MA (US)

(73) Assignee: Novartis AG, Basel (CH)

(21) Appl. No.: 14/681,431

(22) Filed: Apr. 8, 2015

Related U.S. Application Data
(63) Continuation of application No. 13/596,176, filed on Aug. 28, 2012.
Application No. 13/596,176 is a continuation of application No. 12/539,855, filed on Aug. 12, 2009, now abandoned.

(51) Int. Cl. A61K 31/5377; A61K 31/501; A61K 45/06; A61K 31/506

(52) U.S. Cl. CPC A61K 31/5377; A61K 31/506; A61K 31/501; A61K 45/06

(57) ABSTRACT

A combination of a BCR-ABL inhibitor and a hedgehog pathway inhibitor for the treatment of leukemia.

This is a continuation of application Ser. No. 12/539,855 filed on Aug. 12, 2009, which in its entirety is herein incorporated by reference.

### BACKGROUND OF THE INVENTION

1. Field of the Invention

A combination of a BCR-ABL inhibitor and a hedgehog pathway inhibitor for the treatment of leukemia.

2. Related Background Art

The Hedgehog signaling pathway has been described in the art (see, e.g., Nybakken et al., Curr. Opin. Genet. Dev. 2002, 12:503-511; and Lum et al., Science 2003, 299: 2039-2045). Briefly, in the absence of hedgehog ligands, the transmembrane receptor, Patched (Ptch), binds to Smoothened (Smo) and blocks Smo's function. This inhibition is relieved in the presence of ligands, which allows Smo to initiate a signaling cascade that results in the release of transcription factors Glis from cytoplasmic proteins fused (Fu) and Suppressor of Fused (SuFu). In the inactive situation, SuFu prevents Glis from translocating to the nucleus. In the active situation, Fu inhibits SuFu and Glis are released. Gli proteins translocate into the nucleus and control target gene transcription.

The BCR-ABL oncogene is the product of Philadelphia chromosome (Ph) 22q, and encodes a chimeric BCR-ABL protein that has constitutively activated ABL tyrosine kinase activity. (Lugo et al., Science 1990, 247:1079-1082). BCR-ABL is the underlying cause of chronic myeloid leukemia (aka chronic myelogenous leukemia or CML). Whereas the 210 kDa BCR-ABL protein is expressed in patients with CML, a 190 kDa BCR-ABL protein resulting from an alternative breakpoint in the BCR gene is expressed in patients with Ph positive (Ph⁺) acute lymphoblastic leukemia (ALL). (Bartram et al., Nature 1983, 306:277-280; Chan et al., Nature 1987, 325:635-637).

BCR-ABL has been shown to induce proliferation and anti-apoptosis through various mechanisms in committed myeloid or lymphoid progenitors or 3T3 fibroblasts. (Pendergast et al., Cell 1993, 75:175-85; Ilaria et al., J. Biol. Chem. 1996, 371:31704-10; Chai et al., J. Immunol. 1997, 159:4720-8; and Skorski et al., EMBO J. 1997, 1.6:6151-61). However, little is known about the effect of BCR-ABL on the hematopoietic stem cell (HSC) population. Recent publications suggest that developmental pathways like the Wnt signaling pathway or the Polycomb gene BMI1 might be involved in the regulation and expansion of leukemic stem cells (Mohty et al., Blood, 2007; Hosen et al., Stem Cells, 2007). BMI1 and beta-catenin are both upregulated in CML blast crisis and their expression correlates with the progression of the disease. BCR-ABL positive granulocyte-macrophage progenitors that have acquired β-catenin expression are candidate leukemic stem cells in blast-crisis CML. Self-renewal pathways are involved in the expansion of the BCR-ABL positive leukemic stem cell during chronic phase, which leads to the initial expansion of the malignant clone.

### BRIEF SUMMARY OF THE INVENTION

The invention provides combinations and therapeutic methods of treatment which may be useful for inhibiting tumor cell growth and for treating a variety of cancers.

In one aspect, the present invention provides a combination comprising a first agent that inhibits the hedgehog signaling pathway and a second agent that inhibits BCR-ABL. In another aspect, the invention provides pharmaceutical compositions comprising a therapeutically effective amount of a first agent that inhibits hedgehog signaling pathway, a second agent that inhibits BCR-ABL, and a pharmaceutically acceptable carrier.

The invention also provides methods for treating cancers, particularly a BCR-ABL positive leukemia, such as CML, comprising administering to a system or a subject, a therapeutically effective amount of a composition comprising a first agent that inhibits hedgehog signaling pathway and a second agent that inhibits BCR-ABL, or pharmaceutically acceptable salts or pharmaceutical compositions thereof, thereby treating said BCR-ABL positive leukemia. For example, the compositions of the invention may be used to treat chronic myeloid leukemia or acute lymphocyte leukemia.

Furthermore, the present invention provides for the use of a therapeutically effective amount of a combination comprising a first agent that inhibits hedgehog signaling pathway and a second agent that inhibits BCR-ABL, or pharmaceutically acceptable salts or pharmaceutical compositions thereof, in the manufacture of a medicament for treating a cell proliferative disorder, particularly BCR-ABL positive leukemia.

In the above compositions and methods for using the compositions of the invention, the first agent in the inventive composition may bind to Smo. In other embodiments, the second agent in the inventive composition is an ABL inhibitor, an ABL/Scr inhibitor, an Aurora kinase inhibitor, or a non-ATP competitive inhibitor of BCR-ABL.

In the above combinations, compositions and methods for using the compositions of the invention, the inventive composition may be administered to a system comprising cells or tissues. In some embodiments, the invention composition may be administered to a human patient or animal subject.

### BRIEF DESCRIPTION OF THE DRAWINGS

FIG. 1 shows a “first replate” experiment in which the total number of secondary colonies derived from a single colony of primary CML cells formed after 3 days treatment with increasing concentrations of Compound A. Results are expressed as a percentage of untreated control.

FIG. 2 shows total colonies of primary CML cells formed following a “second replate” of FIG. 1. Results are expressed as a percentage of untreated control shows total colonies formed following a second replate.

FIG. 3 describes the total numbers of resultant secondary colonies as a percentage of the untreated control in three replicates.

FIG. 4 is an illustrative example of the total numbers of secondary colonies in a experiment of primary CML cells treated with compound A or nilotinib or the two drugs in combination for 3 days. Results are expressed as a percentage of the untreated control.

FIG. 5 indicates the total number of secondary colonies produced in “first replate” experiments following 7 days exposure to compound A, nilotinib or a combination of the two. Results are expressed as a percentage of the untreated control.

FIG. 6 indicates the total number of secondary colonies produced in “first replate” experiments following 3 days exposure to compound A, nilotinib or a combination of the two. Results are expressed as a percentage of the untreated control.

FIG. 7 demonstrates the proliferation index (PI) of primary CML cells after treatment with compound A, nilotinib or both drugs by calculating the area under the curve (AUC) for the assays. The PI reflects both the colonies produced and their extinction rate.

FIG. 8 is 2.5×10⁵ mouse bone marrow cells infected with Bcr-abl retrovirus were plated in 400 ul per well (48-well plate) in OPTI-MEM media (10% FBS, 0.1% 2-Mercaptoethanol, 50 ng/ml SCF, 25 ng/ml mIL-3 and 25 ng/ml mIL-6) in the presence of the indicated concentrations of AMN107 and compound A. After 3 days of culture cells were plated in methylcellulose at a concentration of 1500 cells per 35 mm plate. Colony formation was scored 10 days after plating.

FIG. 9. Colonies obtained in the 1^{st} plating experiment of FIG. 8 were resuspended and washed in PBS containing 10% FCS. Cells were resuspended in OPTI-MEM media and plated in methylcellulose at a concentration of 5000 cells per 35 mm plate. Colony formation was scored 10 days after plating.

FIG. 10 compares survival rates in a mouse CML model with a control vehicle. Compound A, AMN107, and a combination of Compound A and ANM107.

### DETAILED DESCRIPTION OF THE INVENTION

The present invention is further exemplified, but not limited, by the following representative examples, which are intended to illustrate the invention and are not to be construed as being limitations thereon.

### Compounds for Formula I—Smoothened Inhibitors

In one aspect, the present invention provides a compound of Formula I:

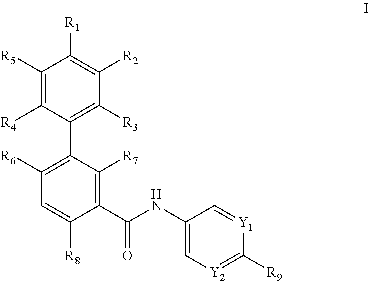

in which

Y₁ and Y₂ are independently selected from N and CR₁₀; wherein R₁₀ is selected from hydrogen, halo, C₁₋₆alkyl, halosubstituted-C₁₋₆alkyl, C₁₋₆alkoxy, halosubstituted-C₁₋₆alkoxy and —OXNR₁₀ₐR_{10b}; wherein R₁₀ₐ and R_{10b} are independently selected from hydrogen and C₁₋₆alkyl;

R₁ is selected from cyano, halo, C₁₋₆alkyl, halosubstituted-C₁₋₆alkyl, C₁₋₆alkoxy; halosubstituted-C₁₋₆alkoxy, C₆₋₁₀aryl, dimethyl-amino, C₁₋₆alkyl-sulfanyl and C₃₋₈heterocycloalkyl optionally substituted with up to 2 C₁₋₆alkyl radicals;

R₂ and R₅ are independently selected from hydrogen, cyano, halo, C₁₋₆alkyl, halosubstituted-C₁₋₆alkyl, C₁₋₆alkoxy, halosubstituted-C₁₋₆alkoxy and dimethylamino;

R₃ and R₄ are independently selected from hydrogen, halo, cyano, C₁₋₆alkyl, halosubstituted-C₁₋₆alkyl, C₁₋₆alkoxy and halosubstituted-C₁₋₆alkoxy; or either R₁ and R₂ or R₁ and R₅ together with the phenyl to which they are both attached form C₅₋₁₀heteroaryl;

R₆ and R₇ are independently selected from hydrogen, C₁₋₆alkyl, halosubstituted-C₁₋₆alkyl, C₁₋₆alkoxy and halosubstituted-C₁₋₆alkoxy; with the proviso that R₆ and R₇ are not both hydrogen;

R₈ is selected from hydrogen, halo, C₁₋₆alkyl, halosubstituted-C₁₋₆alkyl, C₁₋₆alkoxy and halosubstituted-C₁₋₆alkoxy;

R₉ is selected from —S(O)₂R₁₁, —C(O)R₁₁, —OR₁₁, —NR₁₂ₐR_{12b} and —R₁₁; wherein R₁₁ is selected from aryl, heteroaryl, cycloalkyl and heterocycloalkyl; R₁₂ₐ and R_{12b} are independently selected from C₁₋₆alkyl and hydroxy-substituted-C₁₋₆alkyl;

wherein said aryl heteroaryl, cycloalkyl and heterocycloalkyl of R₉ can be optionally substituted with 1 to 3 radicals independently selected from C₁₋₆alkyl, halosubstituted-C₁₋₆-alkyl, C₁₋₆alkoxy, halosubstituted-C₁₋₆alkoxy, C₆₋₁₀aryl-C₀₋₄alkyl, C₅₋₁₀heteroaryl-C₀₋₄alkyl, C₃₋₁₂cycloalkyl and C₃₋₈heterocycloalkyl;

wherein said aryl-alkyl substituent of R₉ is optionally substituted with 1 to 3 radicals independently selected from halo, C₁₋₆alkyl, halosubstituted-C₁₋₆alkyl, C₁₋₆alkoxy, halosubstituted-C₁₋₆alkoxy and methyl-piperazinyl; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

Compounds of Formula I are hedgehog pathway inhibitors.

Preferred compounds of Formula I are selected from 4′-cyano-6-methyl-biphenyl-3-carboxylic acid [4-(morpholine-4-sulfonyl)-phenyl]-amide, 4′-cyano-6-methyl-biphenyl-3-carboxylic acid [6-(2,6-dimethyl-morpholin-4-yl)-pyridin-3-yl]-amide, 4′-Cyano-2-methyl-biphenyl-3-carboxylic acid (6-azepan-1-yl-pyridin-3-yl)-amide, 4′-Methoxy-2-methyl-biphenyl-3-carboxylic acid (6-azepan-1-yl-pyridin-3-yl)-amide, 4′-Methoxy-2-methyl-biphenyl-3-carboxylic acid (4-cyclohexyl-phenyl)-amide, 4′-Methoxy-2-methyl-biphenyl-3-carboxylic acid [6-(2-methyl-morpholin-4-yl)-pyridin-3-yl]-amide, 4′-Dimethylamino-2-methyl-biphenyl-3-carboxylic acid (4-cyclohexyl-phenyl)-amide, 4′-Dimethylamino-2-methyl-biphenyl-3-carboxylic acid (4-morpholin-4-yl-phenyl)-amide, 6-Chloro-4′-dimethylamino-biphenyl-3-carboxylic acid (6-[1,4]oxazepan-4-yl-pyridin-3-yl)-amide, 6-Chloro-4′-dimethylamino-biphenyl-3-carboxylic acid (6-morpholin-4-yl-pyridin-3-yl)-amide, 6-Chloro-4′-dimethylamino-biphenyl-3-carboxylic acid (6-azepan-1-yl-pyridin-3-yl)-amide, 6-Chloro-4′-methoxy-biphenyl-3-carboxylic acid [6-(2-methyl-morpholin-4-yl)-pyridin-3-yl]-amide, 6-Chloro-4′-methoxy-biphenyl-3-carboxylic acid (6-[1,4]oxazepan-4-yl-pyridin-3-yl)-amide, 6-Chloro-4′-methoxy-biphenyl-3-carboxylic acid (6-azepan-1-yl-pyridin-3-yl)-amide, 6-Chloro-4′-methoxy-biphenyl-3-carboxylic acid (6-morpholin-4-yl-pyridin-3-yl)-amide, 4′-Methoxy-6-methyl-biphenyl-3-carboxylic acid (6-morpholin-4-yl-pyridin-3-yl)-amide, 4′-Methoxy-6-methyl-biphenyl-3-carboxylic acid (6-[1,4]oxazepan-4-yl-pyridin-3-yl)-amide, 4′-Methoxy-6-methyl-biphenyl-3-carboxylic acid [6-(2-methyl-morpholin-4-yl)-pyridin-3-yl]-amide, 4′-Dimethylamino-6-methyl-biphenyl-3-carboxylic acid [6-(2-methyl-morpholin-4-yl)-pyridin-3-yl]-amide, 4′-Dimethylamino-6-methyl-biphenyl-3-carboxylic acid (6-[1,4]oxazepan-4-yl-pyridin-3-yl)-amide, 4′-Dimethylamino-6-methyl-biphenyl-3-carboxylic acid (6-morpholin-4-yl-pyridin-3-yl)-amide, 4′-Methoxy-6-methyl-biphenyl-3-carboxylic acid (6-azepan-1-yl-pyridin-3-yl)-amide, 4′-Ethoxy-6-methyl-biphenyl-3-carboxylic acid (6-azepan-1-yl-pyridin-3-yl)-amide, 6-Methyl-4′-methylsulfanyl-biphenyl-3-carboxylic acid (6-azepan-1-yl-pyridin-3-yl)-amide, 4′-Dimethylamino-6-methyl-biphenyl-3-carboxylic acid (6-azepan-1-yl-pyridin-3-yl)-amide, 6-Methyl-[1,1′;4′1″]terphenyl-3-carboxylic acid (6-azepan-1-yl-pyridin-3-yl)-amide, 3′-Chloro-6-methyl-biphenyl-3-carboxylic acid (6-azepan-1-yl-pyridin-3-yl)-amide, 2′,4′-Dichloro-6-methyl-biphenyl-3-carboxylic acid (6-azepan-1-yl-pyridin-3-yl)-amide, 2′-Chloro-6-methyl-biphenyl-3-carboxylic acid (6-azepan-1-yl-pyridin-3-yl)-amide, 3′-Chloro-6-methyl-biphenyl-3-carboxylic acid (6-azepan-1-yl-pyridin-3-yl)-amide, 3′,4′-Dichloro-6-methyl-biphenyl-3-carboxylic acid (6-azepan-1-yl-pyridin-3-yl)-amide, 3′-Chloro-6-methyl-4′-trifluoromethyl-biphenyl-3-carboxylic acid (6-azepan-1-yl-pryridin-3-yl)-amide, 6,4′-Dimethyl-biphenyl-3-carboxylic acid (6-azepan-1-yl-pyridin-3-yl)-amide, 4′-Ethyl-6-methyl-biphenyl-3-carboxylic acid (6-azepan-1-yl-pyridin-3-yl)-amide, 4′-tert-Butyl-6-methyl-biphenyl-3-carboxylic acid (6-azepan-1-yl-pyridin-3-yl)-amide, 6-Methyl-4′-propyl-biphenyl-3-carboxylic acid (6-azepan-1-yl-pyridin-3-yl)-amide, 4′-Isobutyl-6-methyl-biphenyl-3-carboxylic acid (6-azepan-1-yl-pyridin-3-yl)-amide, 4′-Isopropyl-6-methyl-biphenyl-3-carboxylic acid (6-azepan-1-yl-pyridin-3-yl)-amide, 6,2′,6′-Trimethyl-biphenyl-3-carboxylic acid (6-azepan-1-yl-pyridin-3-yl)-amide, 6,2′,3′-Trimethyl-biphenyl-3-carboxylic acid (6-azepan-1-yl-pyridin-3-yl)-amide, 6-Methyl-4′-trifluoromethyl-biphenyl-3-carboxylic acid (6-azepan-1-yl-pyridin-3-yl)-amide, 6-Methyl-3′-trifluoromethyl-biphenyl-3-carboxylic acid (6-azepan-1-yl-pyridin-3-yl)-amide, 6-Methyl-3′, 5′-bistrifluoromethyl-biphenyl-3-carboxylic acid (6-azepan-1-yl-pyridin-3-yl)-amide, 3′-Isopropoxy-6-methyl-biphenyl-3-carboxylic acid (6-azepan-1-yl-pyridin-3-yl)-amide, 3′-Ethoxy-6-methyl-biphenyl-3-carboxylic acid (6-azepan-1-yl-pyridin-3-yl)-amide, 2′,6′-Dimethoxy-6-methyl-biphenyl-3-carboxylic acid (6-azepan-1-yl-pyridin-3-yl)-amide, 6-Methyl-4′-trifluoromethoxy-biphenyl-3-carboxylic acid (6-azepan-1-yl-pyridin-3-yl)-amide, 6-Methyl-3′-trifluoromethoxy-biphenyl-3-carboxylic acid (6-azepan-1-yl-pyridin-3-yl)-amide, 6-Methyl-biphenyl-3-carboxylic acid (4-morpholin-4-yl-phenyl)-amide, 4′-Methoxy-6-methyl-biphenyl-3-carboxylic acid (4-morpholin-4-yl-phenyl)-amide, 3′-Methoxy-6-methyl-biphenyl-3-carboxylic acid (4-morpholin-4-yl-phenyl)-amide, 4′-(2-Dimethylamino-ethoxy)-6-methyl-biphenyl-3-carboxylic acid (4-morpholin-4-yl-phenyl)-amide, 3′-Dimethylamino-6-methyl-biphenyl-3-carboxylic acid (4-morpholin-4-yl-phenyl)-amide, 4′-Fluoro-6-methyl-biphenyl-3-carboxylic acid (4-morpholin-4-yl-phenyl)-amide, 3′-Fluoro-6-methyl-biphenyl-3-carboxylic acid (4-morpholin-4-yl-phenyl)-amide, 2′-Fluoro-6-methyl-biphenyl-3-carboxylic acid (4-morpholin-4-yl-phenyl)-amide, 4-Methyl-N-(4-morpholin-4-yl-phenyl)-3-quinoxalin-6-yl-benzamide, 6-Methyl-4′-(4-methyl-piperazin-1-yl)-biphenyl-3-carboxylic acid (4-morpholin-4-yl-phenyl)-amide, 2′-Cyano-6-methyl-biphenyl-3-carboxylic acid (4-morpholin-4-yl-phenyl)-amide, 3′-Cyano-6-methyl-biphenyl-3-carboxylic acid (4-morpholin-4-yl-phenyl)-amide, 4′-Cyano-6-methyl-biphenyl-3-carboxylic acid (6-[1,4]oxazepan-4-yl-pyridin-3-yl)-amide, 4′-Cyano-6-methyl-biphenyl-3-carboxylic acid (6-azepan-1-yl-pyridin-3-yl)-amide, 4′-Cyano-6-methyl-biphenyl-3-carboxylic acid [6-(2-methyl-morpholin-4-yl)-pyridin-3-yl]-amide, 4′-Cyano-6-methyl-biphenyl-3-carboxylic acid (3,4,5,6-tetrahydro-2H-[1,2′]bipyridinyl-5′yl)-amide, 4′-Cyano-6-methyl-biphenyl-3-carboxylic acid (6-morpholin-4-yl-pyridin-3-yl)-amide, 4′-Cyano-6-methyl-biphenyl-3-carboxylic acid [6-(4-methyl-piperazin-1-yl)-pyridin-3-yl]-amide, 4′-Cyano-6-methyl-biphenyl-3-carboxylic acid (4-morpholin-4-yl-phenyl)-amide, 4′-Cyano-6-methyl-biphenyl-3-carboxylic acid (3-fluoro-4-morpholin-4-yl-phenyl)-amide, 4′-Cyano-6-methyl-biphenyl-3-carboxylic acid (3-chloro-4-morpholin-4-yl-phenyl)-amide, 4′-Cyano-6-methyl-biphenyl-3-carboxylic acid (3-bromo-4-morpholin-4-yl-phenyl)-amide, 4′-Cyano-6-methyl-biphenyl-3-carboxylic acid (3-methyl-4-morpholin-4-yl-phenyl)-amide, 4′-Cyano-6-methyl-biphenyl-3-carboxylic acid (4-morpholin-4-yl-3-trifluoromethyl-phenyl)-amide, 4′-Cyano-6-methyl-biphenyl-3-carboxylic acid (4-cyclohexyl-phenyl)-amide, 4′-Cyano-6-methyl-biphenyl-3-carboxylic acid biphenyl-4-ylamide, 4′-Cyano-6-methyl-biphenyl-3-carboxylic acid (4′-methoxy-biphenyl-4-yl)-amide, 4′-Cyano-6-methyl-biphenyl-3-carboxylic acid [4-(4-benzyl-piperazin-1-yl)-phenyl]-amide, 4′-Cyano-6-methyl-biphenyl-3-carboxylic acid [4-(piperidine-1-sulfonyl)-phenyl]-amide, 4′-Cyano-6-methyl-biphenyl-3-carboxylic acid [4-(pyrrolidine-1-sulfonyl)-phenyl]-amide, 4′-Cyano-6-methoxy-biphenyl-3-carboxylic acid (6-azepan-1-yl-pyridin-3-yl)-amide, 4′Cyano-2-methoxy-biphenyl-3-carboxylic acid (6-azepan-1-yl-pyridin-3yl)-amide, 4′-Cyano-2-methyl-biphenyl-3-carboxylic acid (6-azepan-1-yl-pyridin-3-yl)-amide, 3′-Fluoro-4′-methoxy-6-methyl-biphenyl-3-carboxylic acid (6-azepan-1-yl-pyridin-3-yl)-amide, 4′-Isopropoxy-6-methyl-biphenyl-3-carboxylic acid (6-azepan-1-yl-pyridin-3-yl)-amide, 4′-Butoxy-6-methyl-biphenyl-3-carboxylic acid (6-azepan-1-yl-pyridin-3-yl)-amide, 3′-Chloro-4′-methoxy-6-methyl-biphenyl-3-carboxylic acid (6-azepan-1-yl-pyridin-3-yl)-amide, 4′-Methoxy-6,3′-dimethyl-biphenyl-3-carboxylic acid (6-azepan-1-yl-pyridin-3-yl)-amide, 4′-Cyano-2-methyl-biphenyl-3-carboxylic acid [4-(piperidine-1-sulfonyl)-phenyl]-amide, 4′-Cyano-6-fluoro-biphenyl-3-carboxylic acid [4-(piperidine-1-sulfonyl)-phenyl]-amide, 6-Bromo-4′-cyano-biphenyl-3-carboxylic acid [4-(piperidine-1-sulfonyl)-phenyl]-amide, 4′-Cyano-6-methyl-biphenyl-3-carboxylic acid [6-(4-benzyl-[1,4]diazepan-1-yl)-pyridin-3-yl]-amide, 4′Cyano-6-methyl-biphenyl-3-carboxylic acid [6-(4-thiophen-3-ylmethyl-[1,4]diazepan-1-yl)-pyridin-3-yl]-amide, 4′-Cyano-2-methyl-biphenyl-3-carboxylic acid [6-(2,6-dimethyl-morpholin-4-yl)-pyridin-3-yl]amide, 4′-Methoxy-2-methyl-biphenyl-3-carboxylic acid [6-(2,6-dimethyl-morpholin-4-yl)-pyridin-3-yl]-amide, 2-Methyl-4′-trifluoromethyl-biphenyl-3-carboxylic acid [6-(2,6-dimethyl-morpholin-4-yl)-pyridin-3-yl]amide, 2-Methyl-4′-trifluoromethoxy-biphenyl-3-carboxylic acid [6-(2,6-dimethyl-morpholin-4-yl)-pyridin-3-yl]-amide, 4′-Cyano-2-methyl-biphenyl-3-carboxylic acid [6-(2-methyl-morpholin-4-yl)-pyridin-3-yl]-amide, 4′-Cyano-2-fluoro-biphenyl-3-carboxylic acid [4-(piperidine-1-sulfonyl)-phenyl]-amide, 4′-Cyano-6-trifluoromethyl-biphenyl-3-carboxylic acid [4-(piperidine-1-sulfonyl)-phenyl]-amide, 4′-Cyano-6-methyl-biphenyl-3-carboxylic acid [6-(4-pyridin-4-ylmethyl-[1,4]diazepan-1-yl)-pyridin-3-yl]-amide, 4′Cyano-6-methyl-biphenyl-3-carboxylic acid [6-(4-pyridin-3-ylmethyl-[1,4]diazepan-1-yl)-pyridin-3-yl]-amide, 4′Cyano-6-methyl-biphenyl-3-carboxylic acid {6-[4-(2,6-dimethoxy-benzyl)-[1,4]diazepan-1-yl]-pyridin-3-yl}-amide, 4′-Cyano-6-methyl-biphenyl-3-carboxylic acid {6-[4-(2-ethoxy-benzyl)-[1,4]diazepan-1-yl]-pyridin-3-yl}-amide, 4′-Cyano-6-methyl-biphenyl-3-carboxylic acid (6-{4-[2-(4-methyl-piperazin-1-yl)-benzyl]-[1,4]diazepan-1-yl}-pyridin-3-yl)-amide, 4′-Cyano-6-methyl-biphenyl-3-carboxylic acid {6-[4-(4-methoxy-2,3-dimethyl-benzyl)-[1,4]diazepan-1-yl]-pyridin-3-yl}-amide, 4′Cyano-6-methyl-biphenyl-3-carboxylic acid {6-[4-(2,3-dihydro-benzo[1,4]dioxin-6-ylmethyl)-[1,4]diazepan-1-yl]-pyridin-3-yl}-amide, 4′Cyano-6-methyl-biphenyl-3-carboxylic acid [6-(4-pyridin-2-ylmethyl-[1,4]diazepan-1-yl)-pyridin-3-yl]-amide, 4′-Cyano-6-methyl-biphenyl-3-carboxylic acid [6-(4-benzo[1,3]dioxol-4-ylmethyl-[1,4]diazepan-1-yl)-pyridin-3-yl]amide, 4′Cyano-6-methyl-biphenyl-3-carboxylic acid {6-[4-(2-trifluoromethoxy-benzyl)-[1,4]diazepan-1-yl]-pyridin-3-yl}-amide, 4′-Cyano-6-methyl-biphenyl-3-carboxylic acid {6-[4-(2-dimethylamino-benzyl)-[1,4]diazepan-1-yl]-pyridin-3-yl}-amide, 4′-Cyano-6-methyl-biphenyl-3-carboxylic acid {6-[4-(2-chloro-5-trifluoromethyl-benzyl)-[1,4]diazepan-1-yl]-pyridin-3-yl}-amide, 4′Cyano-6-methyl-biphenyl-3-carboxylic acid {6-[4-(2,3-difluoro-benzyl)-[1,4]diazepan-1-yl]-pyridin-3-yl}-amide, 4′-Cyano-6-methyl-biphenyl-3-carboxylic acid {6-[4-(2-chloro-4-fluoro-benzyl)-[1,4]diazepan-1-yl]-pyridin-3-yl}-amide, 4′Cyano-6-methyl-biphenyl-3-carboxylic acid {6-[4-(2,6-difluoro-benzyl)-[1,4]diazepan-1-yl]-pyridin-3-yl}-amide, 2-Chloro-4′-cyano-biphenyl-3-carboxylic acid [4-(piperidine-1-sulfonyl)-phenyl]-amide, 4′-Cyano-6-trifluoromethyl-biphenyl-3-carboxylic acid [6-(2,6-dimethyl-morpholin-4-yl)-pyridin-3-yl]-amide, 2-Chloro-4′-cyano-biphenyl-3-carboxylic acid [6-(2,6-dimethyl-morpholin-4-yl)-pyridin-3-yl]-amide, 4′-Cyano-6-ethyl-biphenyl-3-carboxylic acid [6-(2,6-dimethyl-morpholin-4-yl)-pyridin-3-yl]-amide, 4′-Cyano-6-methyl-biphenyl-3-carboxylic acid {6-[4-(3-fluoro-benzyl)-piperazin-1-yl]-pyridin-3-yl}-amide, 4′-Cyano-6-methyl-biphenyl-3-carboxylic acid {6-[4-(2-trifluoromethoxy-benzyl)-piperazin-1-yl]-pyridin-3-yl}-amide, 4′Cyano-6-methyl-biphenyl-3-carboxylic acid {6-[4-(3-chloro-benzyl)-piperazin-1-yl]-pyridin-3-yl}-amide, 4′-Cyano-6-methyl-biphenyl-3-carboxylic acid {6-[4-(4-isobutyl-benzyl)-piperazin-1-yl]-pyridin-3-yl}-amide, 4′-Cyano-6-methyl-biphenyl-3-carboxylic acid {6-[4-(4-tert-butyl-benzyl)-piperazin-1-yl]-pyridin-3-yl}-amide, 4′Cyano-6-methyl-biphenyl-3-carboxylic acid {6-[4-(7-methoxy-benzo[1,3]dioxol-5-ylmethyl)-piperazin-1-yl]pyridin-3-yl}-amide, 4′-Cyano-6-methyl-biphenyl-3-carboxylic acid [6-(4-benzyl-piperazin-1-yl)-pyridin-3-yl]-amide, 4′-Cyano-6-methyl-biphenyl-3-carboxylic acid [6-(4-pyridin-3-ylmethyl-piperazin-1-yl)-pyridin-3-yl]-amide, 4′-Cyano-6-methyl-biphenyl-3-carboxylic acid {6-[4-(4-difluoromethoxy-benzyl)-piperazin-1-yl]-pyridin-3-yl}-amide, 4′Cyano-6-methyl-biphenyl-3-carboxylic acid {6-[4-(4-cyano-benzyl)-piperazin-1-yl]-pyridin-3-yl}-amide, 4′-Cyano-6-methyl-biphenyl-3-carboxylic acid [6-(4-quinolin-5-ylmethyl-piperazin-1-yl)-pyridin-3-yl]-amide, 4′-Cyano-6-methyl-biphenyl-3-carboxylic acid [6-(4-pyridin-4-ylmethyl-piperazin-1-yl)-pyridin-3-yl]-amide, 4′-Cyano-6-methyl-biphenyl-3-carboxylic acid [6-(4-pyridin-2-ylmethyl-piperazin-1-yl)-pyridin-3-yl]-amide, 4′-Cyano-6-methyl-biphenyl-3-carboxylic acid {6-[4-(4-imidazol-1-yl-benzyl)-piperazin-1-yl]-pyridin-3-yl}-amide, 4′-Cyano-6-methyl-biphenyl-3-carboxylic acid {6-[4-(3-cyano-benzyl)-piperazin-1-yl]-pyridin-2-yl}-amide, 4′Cyano-6-methyl-biphenyl-3-carboxylic acid [6-(4-isoquinolin-5-ylmethyl-piperazin-1-yl)-pyridin-3-yl]-amide, (R)-2-methyl-N-(6-(2-methylmorpholino)pyridin-3-yl)-4′-(trifluoromethoxy)biphenyl-3-carboxamide, 4′-cyano-2-methyl-N-(6-sulfonylmorpholinopyridin-3-yl)biphenyl-3-carboxamide, (S)-4′-cyano-2-methyl-N-(6-(2-methylmorpholino)pyridin-3-yl)biphenyl-3-carboxamide, (R)-6-chloro-N-(6-(2-methylmorpholino)pyridin-3-yl)-4′-(trifluoromethoxy)biphenyl-3-carboxamide, 4′-cyano-2-methyl-N-(6-sulfinylmorpholinopyridin-3-yl)biphenyl-3-carboxamide, 4′-cyano-N-(6-(diisobutylamino)pyridin-3-yl)-2-methylbiphenyl-3-carboxamide, 4′-cyano-N-(2-((2S,6R)-2,6-dimethylmorpholino)pyrimidin-5-yl)-2-methylbiphenyl-3-carboxamide, N-(2-((2S,6R)-2,6-dimethylmorpholino)pyrimidin-5-yl)-2-methyl-4′-(trifluoromethyl)biphenyl-3-carboxamide, N-(2-((2S,6R)-2,6-dimethylmorpholino)pyrimidin-5-yl)-2-methyl-4′-(trifluoromethoxy)biphenyl-3-carboxamide, N-(2-bis(2-hydroxyethyl)amino)pyrimidin-5-yl)-2-methyl-4′-(trifluoromethoxy)biphenyl-3-carboxamide, 2-methyl-N-(6-(tetrahydro-2H-pyran-4-yloxy)pyridin-3-yl)-4′-(trifluoromethoxy)biphenyl-3-carboxamide, N-(5-chloro-6-((2S,6R)-2,6-dimethylmorpholino)pyridin-3-yl)-2-methyl-4′-(trifluoromethoxy)biphenyl-3-carboxamide, N-(6-((2R,6S)-2,6-dimethyltetrahydro-2H-pyran-4-yl)pyridin-3-yl)-2-methyl-4′-(trifluoromethoxy)biphenyl-3-carboxamide,
N-(6-(4-ethylpiperazine-1-carbonyl)pyridin-3-yl)-2-methyl-4-′-(trifluoromethoxy)biphenyl-3-carboxamide, 2-methyl-N-(6-(2-oxopiperazin-1-yl)pyridin-3-yl)-4′-(trifluoromethoxy)biphenyl-3-carboxamide, 2-methyl-N-(6-(1-(pyridin-4-ylmethyl)piperidin-4-yl)pyridin-3-yl)-4′-(trifluoromethoxy)biphenyl-3-carboxamide, 2-methyl-N-(6-(2-oxo-4-(pyridin-4-ylmethyl)piperazin-1-yl)pyridin-3-yl)-4′-(trifluoromethoxy)biphenyl-3-carboxamide, 2-methyl-N-(6-(1-pyridin-4-ylmethyl)piperidin-3-yl)pyridin-3-yl)-4′-(trifluoromethoxy)biphenyl-3-carboxamide, N-(6-(1-ethylpiperidin-3-yl)pyridin-3-yl)-2-methyl-4′-(trifluoromethoxy)biphenyl-3-carboxamide and N-(6-((2R,6S)-2,6-dimethylmorpholino)pyridin-3-yl)-2-methyl-4′-(trifluoromethoxy)biphenyl-3-carboxamide and 2-Methyl-4′-trifluoromethoxy-biphenyl-3-carboxylic acid [6-(cis-2,6-dimethyl-morpholin-4-yl)-pyridin-3-yl]-amide, (also identified as Compound A in this document), which has the formula:

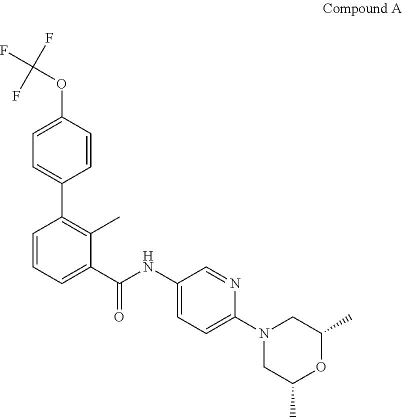 [SMILES: Cc1c(C(=O)Nc2ccc(N3C[C@@H](C)O[C@@H](C)C3)nc2)cccc1-c1ccc(OC(F)(F)F)cc1]

The above compounds of Formula I are further described in WO 2007/131201.

### Compounds of Formula—Smoothened Inhibitors

The present invention relates to a compounds of the formula (II):

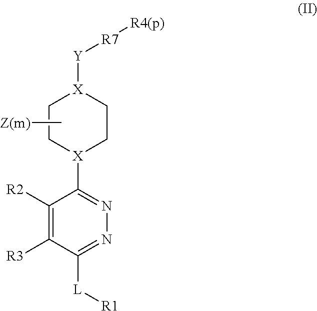

and pharmaceutically acceptable salts thereof, wherein

R1 is a C₆₋₁₄ aryl group, or a 5-14 membered heteroaryl group which may be unsubstituted or substituted;

R2 and R3 are independently C₁₋₈alkyl, C₁₋₈alkylOH, or R2 and R3 form a fused C₃₋₁₄ cycloalkyl group;

L is a bond, C₁₋₈ alkylene, —C(O)O—, —C(O)NR9—, —C₁₋₈ alkylOH—, —C₁₋₈ haloalkyl-, —C(O)—, —NH—or —O—;

X and W are independently N or CR5, and at least one of X or W is N;

R7is a C₆₋₁₄ aryl group, a 5-14 membered heteroaryl group, or a 3-14 membered cycloheteroalkyl group;

R4 is C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ alkoxy, halo, NR6R8, C(O)OR6, C(O)NR6R8, C₁₋₈haloalkyl, formyl, carbalkoxy, C₁₋₈alkylOH, C(O)R6, SO₂R6, C(O)NHC₁₋₈alkylR6, NR6R8, SO₂NR6R8, OCF₃, NHC(O)R6, CH₂OC(O)NR6R8, CH₂NR6R8, NHC(O)OR6, NHC(O)NR6R8, CH₂NHSO₂R6, CH₂NHC(O)OR6, OC(O)R6, or NHC(O)R6, which may be substituted or unsubstituted;

Z is C₁₋₈ alkyl, CN, OH, or halogen;

m and p are independently 0-3;

Y is a bond, C₁₋₈ alkylene, —C(O)—, —C(O)O—, —CH(OH)—, or —C(O)NR10;

R5 is H, halogen, CN, lower alkyl, OH, OCH₃ or OCF₃,

Wherein R1 may be substituted by one or more of C₁₋₈ alkyl, a C₆₋₁₄ aryl group, C₁₋₈ haloalkyl, C₁₋₈ alkoxy, halo, NH₂, CN, OCF₃, OH, C(O)NR6R8, C(O)R6, NR6R8, NHC(O)R6, SO₂R6, SO₂NR6R8;

R9 and R10 are independently C₁₋₈ alkyl or H;

R6 and R8 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈haloalkyl, C₁₋₈ alkylOH, C₁₋₈alkoxy, or two R6 on one atom can form a heteroatom containing ring; and

Wherein R4, R6, and R8 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH₃—, —OCF₃, —OH, —NH₂.

In another embodiment, the present invention includes compounds of formula (II) wherein R7 is

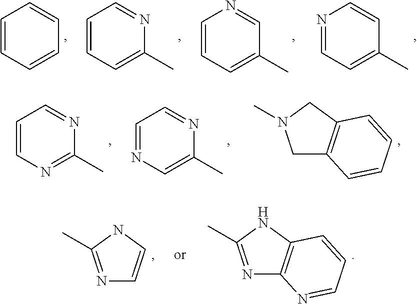

In another embodiment, the present invention includes compounds of formula (II) according to claim 1 wherein R1 is

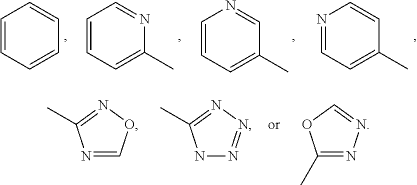

In another embodiment, the present invention includes compounds of formula (II) wherein R7 is

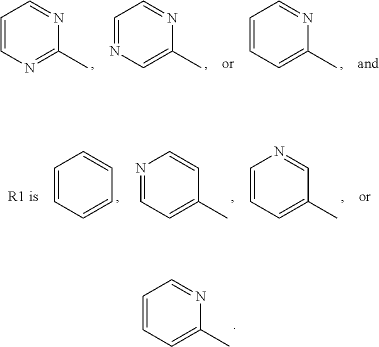

In yet another embodiment, the present invention includes compounds of formula (II) wherein R4 is C(O)OC₁₋₈ alkyl, CF₃, C(O)OR6, C(O)NR6R8, C₁₋₈ haloalkyl, C₁₋₈ alkylOH, C(O)R6, SO₂R6, C(O)NHC₁₋₈ alkylR6, C(CH₃)(CH₃)(OH), C(O)CH₃, C(CH₂)CH₃, or C(CH₃)(CH₂OH)OH; and

R6 and R8 are independently H, C₁₋₈ alkyl, C₁₋₈ alkenyl, C₃₋₁₄ cycloalkyl, a C₆₋₁₄ aryl group, a 5-14membered heteroaryl group, or a 3-14membered cycloheteroalkyl group.

In another embodiment, the present invention includes compounds of formula (II) wherein R4 is

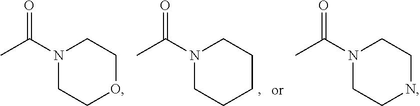

which may be unsubstituted or substituted.

In another embodiment, the present invention includes compounds of formula (II) wherein R2 and R3 are C₁₋₈ alkyl.

In a still further embodiment, the present invention includes compounds of formula (II) wherein R2 and R3 are CH₃.

In another embodiment, the present invention includes compounds of formula (II) wherein L is —O—, —NH—, —C(O)—, —CH(OH)—, —CH₂—, —CF₂—, —CHF—, —COH—, or a bond. In another embodiment, the present invention includes compounds of formula (I) wherein L is —CH₂—. In another embodiment, the present invention includes compounds of formula (I) wherein both X are N, and Z is CH₃.

In another embodiment, the present invention includes a compound of formula (IIa):

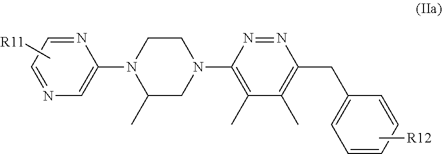

and pharmaceutically acceptable salts thereof, wherein

R11 is C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ alkoxy, halo, NR13R14, C(O)OR13, C(O)NR13R14, C₁₋₈haloalkyl, formyl, carbalkoxy, C₁₋₈alkylOH, C(O)R13, SO₂R13, C(O)NHC₁₋₈alkylR13, NR13R14, SO₂NR13R14, OCF₃, NHC(O)R13, CH₂OC(O)NR13R14, CH₂NR13R14, NCH(O)OR13, NHC(O)NR13R14, CH₂NHSO₂R13, CH₂NHC(O)OR13, OC(O)R13, or NHC(O)R13, which may be substituted or unsubstituted;

R12 is H, C₁₋₈ alkyl, a C₆₋₁₄ aryl group, C₁₋₈ haloalkyl, C₁₋₈ alkoxy, halo, NH₂, CN, OCF₃, OH, C(O)NR13R14, C(O)R13, NR13R14, NHC(O)R13, SO₂R13, SO₂NR13R14;

R13 and R14 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈haloalkyl, C₁₋₈ alkylOH, C₁₋₈alkoxy, or R13and R14 on one atom can form a heteroatom containing ring; and

Wherein R11, R13, and R14 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH₃, —OCF₃, —OH, —NH₂.

Compounds of Formula II and IIa are further described in the contents of U.S. patent application Ser. No. 12/503,565, which has counterpart International Application No. PCT/EP09/059138.

A preferred compound of formula (II) is 2-[(R)-4-(6-Benzyl-4,5-dimethyl-pyridazin-3-yl)-2-methyl-3,4,5,6-tetrahydro-2H-[1,2′]bipyrazinyl-5′-yl]-propan-2-ol, (also identified as Compound B in this document), of the below formula:

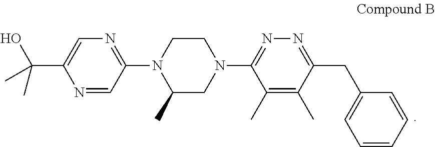 [SMILES: Cc1c(Cc2ccccc2)nnc(N2CCN(c3cnc(C(C)(C)O)cn3)[C@H](C)C2)c1C]

2-[(R)-4-(6-Benzyl-4,5-dimethyl-pyridazin-3-yl)-2-methyl-3,4,5,6-tetrahydro-2H-[1,2′]bipyrazinyl-5′-yl]-propan-2-ol can be made according to Scheme 1

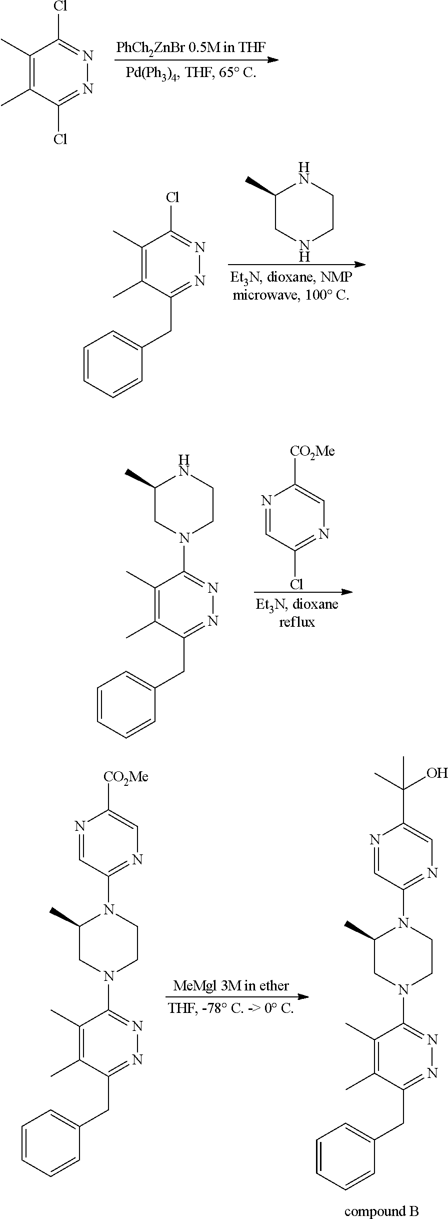

### First Step:

A mixture of 4,5-dimethyl-1,4-dichloro-pyridazine (10 g, 56.5 mmol), tetrakis(triphenylphosphine)palladium(0) (3.3 g, 2.80 mmol) and THF (200 mL) is degassed and then benzylzinc bromide (147 mL, 0.5 M in THF, 73.40 mmol) is added. The reaction solution is heated to 65° C. overnight. Solvent is removed. Water is added and the water layer is extracted with EtOAc. The organic layer is concentrated to afford a crude product that is purified by chromatography on silica gel (EtOAc/Heptane: 0%˜50%) to give 3-benzyl-6-chloro-4,5-dimethyl-pyridazine (9.5 g, 67%).

### Second Step:

3-Chloro-4,5-dimethyl-6-((R)-3-methyl-piperazin-1-yl)-pyridazine (400 mg, 1.66 mmol 1, eq) is added to a solution of benzylzinc bromide (12.3 mL 0.5 M in THF, 6.64 mmol 4 eq) and tetrakis(triphenylphosphine)palladium (100 mg, 0.08 mmol, 0.05 eq) in a microwave vial. The vial is sealed and irradiated in the microwave at 100° C. (high absorption setting) for 40 min. The reaction mixture is concentrated and purified by silica gel chromatography (5-20% EtOAc/heptane) to 3-benzyl-4,5-dimethyl-6-((R)-3-methyl-piperazin-1-yl)-pyridazine (324 mg, 66%).

### Third Step:

A mixture of the above compound (6.0 g, 20.27 mmol), 5-chloropyrazine-2-carboxylic acid methyl ester (5.3 g, 30.30 mmol), Et₃N (6.2 g, 60.60 mmol) and dioxane (100 mL) is heated to reflux overnight. Solvent is removed. Saturated NH₄Cl solution is added and extracted with EtOAc. The organic layer is concentrated to afford the crude product that is purified by chromatography on silica gel (EtOAc/heptane: 50%˜100%) to (R)-4-(6-benzyl-4,5-dimethyl-pyridazin-3-yl)-2-methyl-3,4,5,6-tetrahydro-2H-[1,2′]bipyrazinyl-5′-carboxylic acid methyl ester (6.6 %, 76%) as a yellow solid.

### Final Step:

To a solution of (R)-4-(6-benzyl-4,5-dimethyl-pyridazin-3-yl)-2-methyl-3,4,5,6-tetrahydro-2H-[1,2′]bipyrazinyl-5′-carboxylic acid methyl ester (840 mg, 1.85 mmol) in THF (12 mL) is added methyl magnesium bromide (5 mL, 15 mmol, 3M in ether) at −78° C. The reaction mixture is stirred at 0° C. for 2 h then diluted with DCM and washed with NH₄Cl and water. The combined organic layers are washed with water, brine, dried over Na₂SO₄, filtered and concentrated down. Purification by HPLC of the crude product with acetontrile in water (from 10% to 95% with 3% 1-propanol) at 220 nm wavelength detection provides the desired compound B (400 mg, 50%) next to small amounts of the corresponding methyl ketone. The solvents are removed with a lyophilizer to provide the products as white powders.

### BCR-ABL Inhibitors

Exemplary BCR-ABL inhibitors which may be used to practice the invention, including nilotinib (AMN107), imatinib (STI571), 2,6,9-trisubstituted purine analogs (e.g., AP23464), AZD-0530, bosutinib (SKI-606), CPG070603, pyrido[2,3-d]pyrimidine compounds (e.g., dasatinib (BMS-354825)), PDI66326, PDI73955, PDI80970), ON012380, 3-substituted benzamide derivatives (e.g., INNO-406), MK-0457 (VX-680), PHA-739358, retaspimycin hydrochloride (IPI-504) and GNF-2. (See e.g., Weisberg et al., Nat. Rev. Cancer 2007, supra; Tauchi et al., Int. J. Hematology 2006, 83:294-300; Manley et al., Biochim. Biophys. Acta 2005, supra; Ge et al., J. Med. Chem. 2006, 49:4606-4615; Adrian et al., Nat. Chem. Biol. 2006, 2:95-102; Asaki et al., Bioorg. Med. Chem. Lett. 2006, 16:1421-1425).

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl “Arylene” means a divalent radical derived from an aryl group.

“Heteroaryl” is as defined for aryl above where one or more of the ring members is a heteroatom. For example C₅₋₁₀heteroaryl is a minimum of 5 members as indicated by the carbon atoms but that these carbon atoms can be replaced by a heteroatom. Consequently, C₅₋₁₀heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, pyrrolidinyl-2-one, piperazinyl, piperidinyl, piperidinylone, 1,4-dioxa-8-aza-spiro[4.5]dec-8-yl, thiomorpholino, sulfanomorpholino, sulfonomorpholino, etc.

“Halogen” (or halo) preferably represents chloro or fluoro, but may also be bromo or iodo.

The term “agent” or “test agent” includes any substance, molecule, element, compound, entity, or a combination thereof. It includes, but is not limited to, e.g., protein, polypeptide, small organic molecule, polysaccharide, polynucleotide, and the like. It can be a natural product, a synthetic compound, a chemical compound, or a combination of two or more substances. Unless otherwise specified, the terms “agent”, “substance”, and “compound” can be used interchangeably.

As used herein, “contacting” has its normal meaning and refers to combining two or more molecules (e.g., a small molecule organic compound and a polypeptide) or combining molecules and cells (e.g., a compound and a cell). Contacting can occur in vitro, e.g., combining two or more agents or combining a compound and a cell or a cell lysate in a test tube or other container. Contacting can also occur in a cell or in situ, e.g., contacting two polypeptides in a cell by coexpression in the cell of recombinant polynucleotides encoding the two polypeptides, or in a cell lysate.

The term “hedgehog” is used to refer generically to any member of the hedgehog family, including sonic, indian, desert and tiggy winkle. The term may be used to indicate protein or gene. The term is also used to describe homolog/ortholog sequences in different animal species.

The terms “hedgehog (Hh) signaling pathway” and “hedgehog (Hh) signaling” are used interchangeably and refer to the chain of events normally mediated by various members of the signaling cascade such as hedgehog, patched (Ptch), smoothened (Smo), and Gli. The hedgehog pathway can be activated even in the absence of a hedgehog protein by activating a downstream component. For example, overexpression of Smo will activate the pathway in the absence of hedgehog.

Hh signaling components or members of Hh signaling pathway refer to gene products that participate in the Hh signaling pathway. An Hh signaling component frequently affects the transmission of the Hh signal in cells/tissues, typically resulting in changes in degree of downstream gene expression level and/or phenotypic changes. Hh signaling components, depending on their biological function and effects on the final outcome of the downstream gene activation/expression, may be divided into positive and negative regulators. A positive regulator is an Hh signaling component that positively affects the transmission of the Hh signal, i.e., stimulates downstream biological events when Hh is present. Examples include hedgehog, Smo, and Gli. A negative regulator is an Hh signaling component that negatively affects the transmission of the Hh signal, i.e., inhibits downstream biological events when Hh is present. Examples include (but are not limited to) Ptch and SuFu. Smo is an essential component of the Hh signaling pathway.

Hedgehog signaling antagonists, antagonists of Hh signaling or inhibitors of Hh signaling pathway refer to agents that inhibit the bioactivity of a positive Hh signaling component (such as hedgehog, Ptch, or Gli) or down-regulate the expression of the Hh signaling component. They also include agents which up-regulate a negative regulator of Hh signaling component. A hedgehog signaling antagonist may be directed to a protein encoded by any of the genes in the hedgehog pathway, including (but not limited to) sonic, indian or desert hedgehog, smoothened, ptch-1, ptch-2, gli-1, gli-2, gli-3, etc.

The terms “inhibit,” “inhibiting” or “inhibition,” in the context of modulation of enzymatic activities, inhibition relates to reversible suppression or reduction of an enzymatic activity including competitive, uncompetitive, and noncompetitive inhibition. This can be experimentally distinguished by the effects of the inhibitor on the reaction kinetics of the enzyme, which may be analyzed in terms of the basic Michaelis-Menten rate equation. Competitive inhibition occurs when the inhibitor can combine with the free enzyme in such a way that it competes with the normal substrate for binding at the active site. A competitive inhibitor reacts reversibly with the enzyme to form an enzyme-inhibitor complex [EI], analogous to the enzyme-substrate complex.

The term “subject” includes mammals, especially humans. It also encompasses other non-human animals such as cows, horses, sheep, pigs, cats, dogs, mice, rats, rabbits, guinea pigs, monkeys. The term “patient” refers to a human patient.

The term “treating” includes the administration of compounds or agents to prevent or delay the onset of the symptoms, complications, or biochemical indicia of a disease (e.g., leukemia), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

### Pharmacology and Utility

The combination of the present invention may be used for treating a variety of cancers. In one embodiment, the invention provides an agent that inhibits the hedgehog signaling pathway in combination with an agent that inhibits BCR-ABL, for inhibiting the growth and proliferation of hematopoietic tumors of lymphoid lineage including leukemia, acute lymphocytic leukemia (ALL), acute lymphoblastic leukemia, B-cell lymphoma, T-cell lymphoma, Hodgkins lymphoma, non-Hodgkins lymphoma, hairy cell lymphoma, histiocytic lymphoma, and Burkitts lymphoma; and hematopoietic tumors of myeloid lineage including acute and chronic myelogenous leukemias (CML), myelodysplastic syndrome, myeloid leukemia, and promyelocytic leukemia.

The combination of the present invention are also useful for treating cancers known to be associated with protein tyrosine kinases such as, for example, Src, BCR-ABL and c-kit. In particular embodiments, the combination of the present invention are useful for treating cancers that are sensitive to and resistant to chemotherapeutic agents that target BCR-ABL and c-kit. In particular embodiments, the combination of the present invention may be used for treating BCR-ABL-positive CML and ALL.

Chronic myelogenous leukemia (CML) is a cancer of the bone marrow characterized by increased and unregulated clonal proliferation of predominantly myeloid cells in the bone marrow. Its annual incidence is 1-2 per 100,000 people, affecting slightly more men than women. CML represents about 15-20% of all cases of adult leukemia in Western populations, about 4,500 new cases per year in the U.S. or in Europe. (Faderl et al., N. Engl. J. Med. 1999, 341: 164-72).

CML is a clonal disease that originates from a single transformed hematopoietic stem cell (HSC) or multipotent progenitor cell (MPP) harboring the Philadelphia translocation t(9/22). The expression of the gene product of this translocation, the fusion oncogene BCR-ABL, induces molecular changes which result in expansion of the malignant hematopoiesis including the leukemic stem cell (LSC) pool and the outgrowth and suppression of non-malignant hematopoiesis (Stam et al., Mol Cell Biol. 1987, 7:1955-60). Myeloid cells (granulocytes, monocytes, megakaryocytes, erythrocytes), but also B- and T-cells express BCR-ABL, indicating the MPP or HSC as the start point of the disease. (Fialkow et al., J. Clin. Invest. 1978, 62:815-23; Takahashi et al., Blood 1998, 92:4758-63). In contrast to oncogenes causing AML, like MOZ-TIF2 or MLL-ENL, BCR-ABL does not confer self-renewal properties to committed progenitor cells, but rather utilizes and enhances the self-renewal properties of existing self-renewing cells, like HSCs or MPPs. During the course of the disease, the leukemic stem cell pool expands and in the final stage, the blast crisis, nearly all CD34+CD38− cells carry the Philadelphia translocation.

Imatinib mesylate (STI571, GLEEVEC®) is the standard of therapy for CML with response rates of more than 96%, and works by inhibiting the activity of BCR-ABL. However, despite initial success, patients eventually develop resistance to imatinib mesylate due to acquisition of point mutations in BCR-ABL. In view of the limitations of imatinib mesylate, there is a need for improved methods for treating CML.

In addition, it is contemplated that the combination of the present invention may be used for treating carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine and endocrine pancreatic carcinoma), esophagus, stomach, gall bladder, cervix, thyroid, and skin (including squamous cell carcinoma); tumors of the central and peripheral nervous system including astrocytoma, neuroblastoma, glioma, medulloblastoma and schwannomas; tumors of mesenchymal origin including fibrosarcoma, rhabdomyosarcoma, and osteosarcoma; and other tumors including melanoma, Merkel cell carcinoma, xeroderma pigmentosum, keratoacanthoma, seminoma, thyroid follicular cancer, and teratocarcinoma. It is also contemplated that the combinations of the present invention may be used for treating mastocytosis, germ cell tumors, pediatric sarcomas, and other cancers.

The therapeutic methods described herein may be used in combination with other cancer therapies. For example, Hh antagonists in combination with BCR-ABL inhibitors may be administered adjunctively with any of the treatment modalities, such as chemotherapy, radiation, and/or surgery. For example, they can be used in combination with one or more chemotherapeutic or immunotherapeutic agents; and may be used after other regimen(s) of treatment is concluded. Examples of chemotherapeutic agents which may be used in the compositions and methods of the invention include but are not limited to anthracyclines, alkylating agents (e.g., mitomycin C), alkyl sulfonates, aziridines, ethylenimines, memylmelamines, nitrogen mustards, nitrosoureas, antibiotics, antimetabolites, folic acid analogs (e.g., dihydrofolate reductase inhibitors such as methotrexate), purine analogs, pyrimidine analogs, enzymes, podophyllotoxins, platinum-containing agents, interferons, and interleukins.

Particular examples of known chemotherapeutic agents which may be used in the compositions and methods of the invention include, but are not limited to, busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, acalacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluorouracil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elfornithine, elliptinium acetate, etoglucid, etoposide, flutamide, gallium nitrate, hydroxyurea, interferon-alpha, interferon-beta, interferon-gamma, interleukin-2, lentinan, lonidamine, prednisone, dexamethasone, leucovorin, mitoguazone, mitoxantrone, mopidamol, nitracrine, pentostatin, phenamet, pirarubicin, podophyllinic acid, 2-ethylhydrazide, procarbazine, razoxane, sizofiran, spirogermanium, paclitaxel, tamoxifen, teniposide, tenuazonic acid, triaziquone, 2,2′,2″-trichlorotriethylamine, urethane, vinblastine, vincristine, and vindesine.

The present methods may be used to treat primary, relapsed, transformed, or refractory forms of cancer, including the development of resistance, such as mutations in BCR-ABL leading to resistance. Often, patients with relapsed cancers have undergone one or more treatments including chemotherapy, radiation therapy, bone marrow transplants, hormone therapy, surgery, and the like. Of the patients who respond to such treatments, they may exhibit stable disease, a partial response (i.e., the tumor or a cancer marker level diminishes by at least 50%), or a complete response (i.e., the tumor as welt as markers become undetectable). In either of these scenarios, the cancer may subsequently reappear, signifying a relapse of the cancer.

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A combination of the present invention includes administration at same time as well as sequential administration. A therapeutically effective amount may vary widely depending on the severity of the disease, the age and relative health of the subject, the potency of the compound used and other factors. An indicated daily dosage in the larger mammal, e.g. humans, is in the range from about 10 mg to about 2,500 mg, more preferably about 100 mg to 1000 mg, in dosages such as 100 mg, 200 mg, 400 mg, 500 mg, 600 mg, 700 mg, 800 mg, 900 mg and 1000 mg. These dosages can be conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch, paste, gelatin, tragacanth, methylcellulose, sodium carboxymethycellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also-contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with immunomodulatory or anti-inflammatory substances or other anti-tumor therapeutic agents. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

One of skill in the art will appreciate that the above transformations are only representative of methods for preparation of the compounds of the present invention, and that other well known methods can similarly be used.

### EXAMPLE 1

Primary cells were obtained from newly diagnosed and untreated patients with CML in chronic phase. These cells were enriched for CD34+ using magnetic-activated cell sorting prior to cryopreservation in 10% DMSO and 4% human albumin solution in liquid nitrogen. Samples were thawed and washed in a solution of DNAse, human albumin solution, magnesium chloride, and phosphate buffered saline. Upon thaw primary cells were cultured in a serum free medium comprising of Iscove's Modified Dulbecco Medium with bovine serum albumin, insulin, transferring, β2 mercaptoethanol and growth factors (100 ng/mL Flt3-ligand, 100 ng/mL stem cell factor, 20 ng/mL interleukin (IL)-3, IL-6 and 50 ng/mL granulocyte-colony stimulating factor) for 24 hours.

Viable cells were enumerated using trypan blue dye exclusion and set up in culture in serum free medium (SFM) with the stated concentrations of Compound A and/or nilotinib. Following 72 hours (h) culture the cells were washed twice in phosphate buffered solution (PBS) and viable cells counted, again by trypan blue exclusion. These cells were then used for a series of colony forming and re-plating assays.

In the colony forming assay (CFA) a single cell suspension is created in semisolid media with appropriate cytokines. This enables the assessment of colony formation from each individual cell. In order to measure the effect of compound A and/or nilotinib on the relative abundance of CD34+ CP CML progenitor cells, CFA were set up in METHOCULT with cells plated at an initial concentration of 4000 cells per mL in duplicate. Colonies were identified and enumerated 14-16 days (d) following plating.

An accepted in vitro technique to approximate self-renewal activity is serial re-plating. Colonies derived from a CFA are individually plucked and re-dispersed in further METHOCULT. The capacity to reform colonies following re-dispersion is related to the number of primitive progenitors remaining within each individual colony and is therefore an indirect measurement of self-renewal. Following CFA 20-30 individual non-erythroid colonies from each experimental arm were then plucked with a p10 pipettor (one tip per colony using an inverted microscope) and carefully dispersed into 100 μL METHOCULT with a further 10 μL SFM in 96 well plates prior to incubation for a further 7 d. Resultant secondary colonies were enumerated in each well and; in the case of wells containing secondary colonies; the entire contents were re-dispersed in a further 100 μL METHOCULT to assess tertiary colony formation.

Colony assays were performed in METHOCULT with cells plated at an initial concentration of 4000 cells per mL in duplicate. Colonies were identified and enumerated 14-16 days (d) following plating. 20-30 individual non-erythroid colonies from each experimental arm were then plucked with a p10 pipettor (one tip per colony using an inverted microscope) and carefully dispersed into 100 μL METHOCULT with a further 10 μL SFM in 96 well plates prior to incubation for a further 7 d. Resultant secondary colonies were enumerated in each well and; in the case of wells containing secondary colonies; the entire contents wore re-dispersed in a further 100 μL METHOCULT to assess tertiary colony formation.

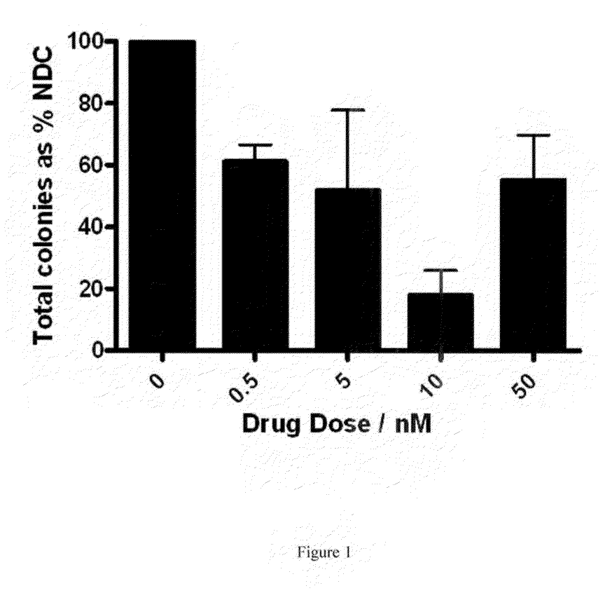

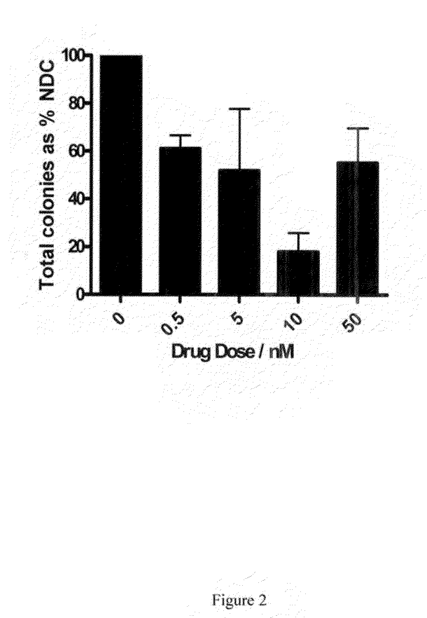

FIG. 1 indicates the total resultant secondary colonies following the first replating as a percentage of the untreated control in three replicates (error bars indicate the standard error of the mean (SEM)). FIG. 2 illustrates the total number of tertiary colonies formed following second replate. These figures indicate a reduction in re-plating capacity with compound A alone and in combination with nilotinib and this is consistent with an inhibition of self-renewal behaviour in the treated cells.

### EXAMPLE 2

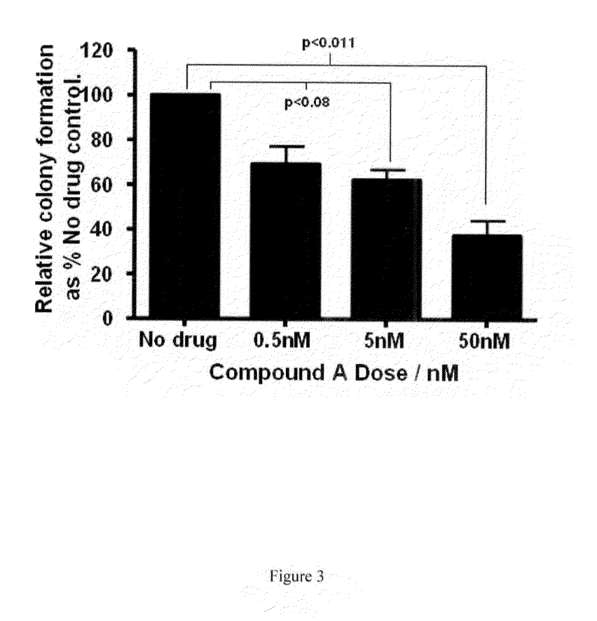

Colony forming assays (CPAs) were performed as described above. Colonies were identified and enumerated 14-16 d following plating. 20-30 individual, non-erythroid colonies from each experimental arm were then plucked as above and carefully dispersed into 100 μL METHOCULT with a further 10 μL SFM in 96 well plates prior to incubation for a further 7 d. Resultant secondary colonies were enumerated to each well. FIG. 3 describes the total numbers of resultant secondary colonies as a percentage of the untreated control in three replicates (significance was assessed by unpaired 2 tailed t test).) and indicates a reduction in re-plating capacity consistent with an inhibition of self-renewal behavior in the treated cells.

### EXAMPLE 3

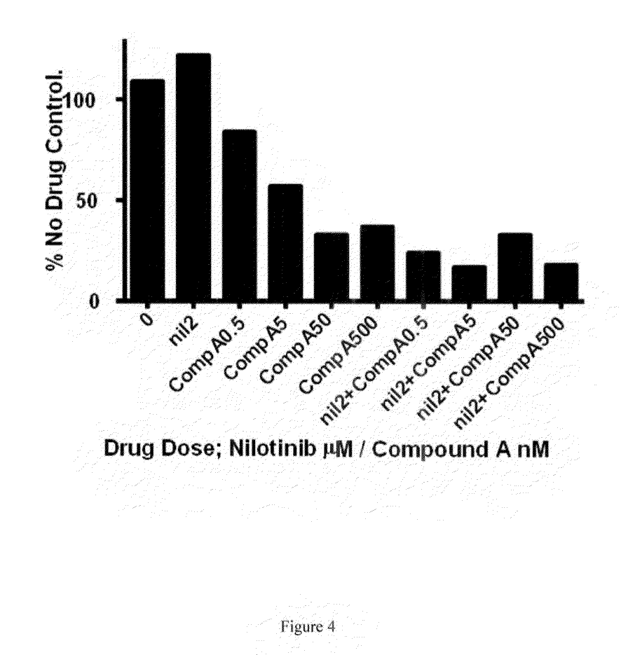

Combination experiments were performed on primary CD34+ selected chronic phase (CP) CML cells. Following thaw and overnight culture primary cells were exposed to Compound A at varying concentrations with or without co-exposure to nilotinib for 72 h in SFM. CFAs and subsequent re-plating experiments were conducted as previously detailed. FIG. 4 details the total numbers of secondary colonies as a percentage of the untreated control in one illustrative example.

### EXAMPLE 4

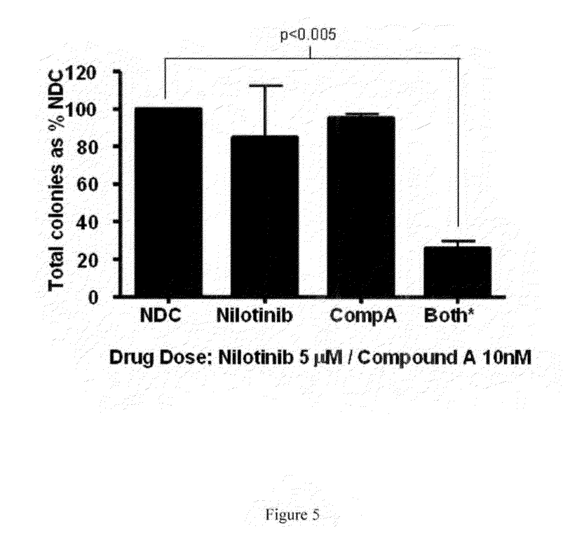

Combination experiments were performed on primary CD34+ selected CP CML cells. Following thaw and overnight culture these primary cells were cultured in Compound A at a final concentration of 10 nM and/or nilotinib at a final concentration of 5 μM for 7 d with CFAs and subsequent re-plating performed as previously indicated. FIG. 5 indicates the total number of secondary colonies produced following re-plating in 3 experiments (error bars indicate the SEM and significance was determined by unpaired 2 tailed t test). FIG. 5 demonstrates a reduction in replating capacity in these cells following exposure to nilotinib and compound A in combination for 7 d.

### EXAMPLE 5

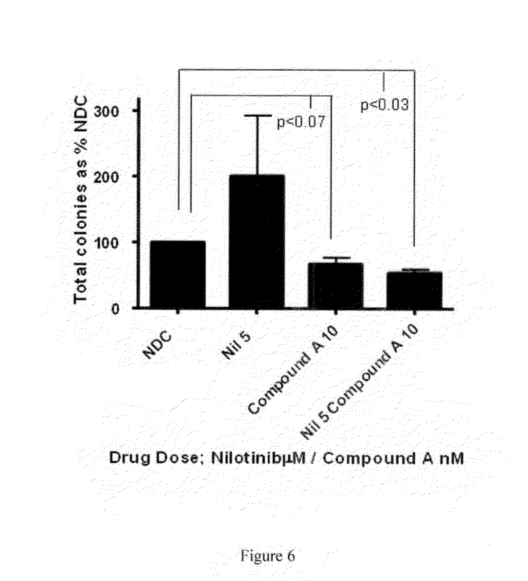

Combination experiments were performed on primary CD34+ selected CP CML cells. Following thaw and overnight culture these primary cells were cultured in Compound A at a final concentration of 10 nM and/or nilotinib at a final concentration of 5 μM for 3 d with CFAs and subsequent re-plating performed as previously indicated. FIG. 6 indicates the total number of secondary colonies produced following re-plating in 4 experiments (error bars indicate the SEM and significance was determined by unpaired 2 tailed t test). FIG. 6 indicates a non-significant increase in re-plating capacity in these cells following 3 d treatment with nilotinib and a reduction in re-plating capacity following 3 d exposure to nilotinib and compound A.

### EXAMPLE 6

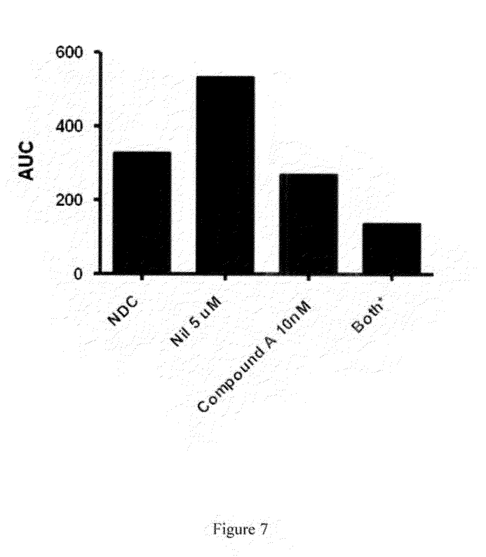

Another measure of the degree of occurring is to assess the proliferation index (PI) of replated colonies. The fate of each re-plated colony is to either become extinct or to produce a number (n) secondary colonies. 20-30 individual non-erythroid colonies from each experimental arm were then plucked with a p10 pipettor (one tip per colony using an inverted microscope) and carefully dispersed into 100 μL METHOCULT with a further 10 μL SFM in 96 well plates prior to incubation for a further 7 d. Resultant secondary colonies were enumerated in each well and; in the case of wells containing secondary colonies; the entire contents were re-dispersed in a further 100 μL METHOCULT to assess tertian colony formation. The PI is a measure of self-renewal that reflects both number of colonies produced and the overall extinction rate. The inverse cumulative distribution of secondary colonies is assessed by graphing the proportion of wells with greater than n colonies and calculating the resultant area under the curve (AUC). In FIG. 7 the PI has been assessed from the resultant totals of all colonies re-plated in each experimental arm is 4 separate experiments (3 d nilotinib and/or compound A exposure) with a total of 132 colonies per arm. This figure demonstrates a relative increase in PI following nilotinib therapy as compared to untreated cells and a reduction in PI following treatment with compound A alone and in combination with nilotinib.

### EXAMPLE 7

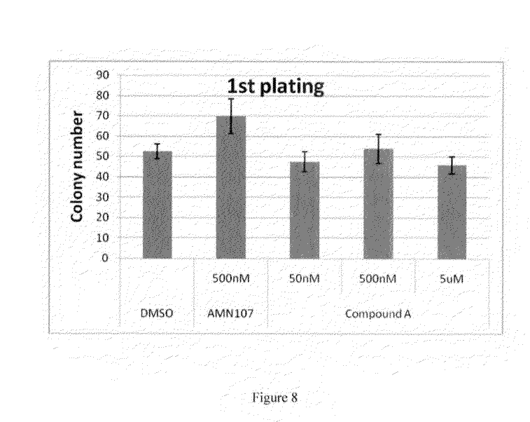

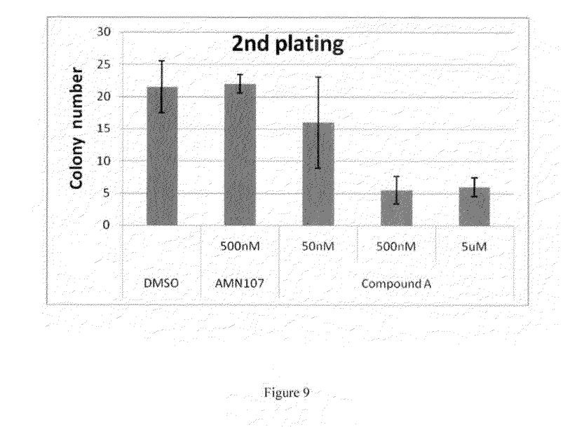

Mouse bone marrow cells were infected with a bicistronic retroviral Bcr-Abl vector (Bcr-Abl-IRES-GFP). Infected bone marrow cells were cultured for 72 h in the presence of cytokines and different concentrations of Compound A or AMN107, and then plated in methylcellulose. No reduction of colony formation was seen in the first methylcellulose plating for the groups pre-treated with AMN107 or Compound A compared to the control DMSO group (FIG. 8). However, a pronounced reduction in colony number was detected in the groups pre-treated with Compound A upon secondary re-plating of the colonies (FIG. 9). Clonogenic colony formation assays assess the self-renewal capacity of early progenitor/stem cells. Data indicate that Compound A inhibits the clonogenic capacity of Bcr-Abl transformed mouse CML bone marrow cells.

### EXAMPLE 8

A bone marrow transplant model of Bcr-Abl was used to induce CML in mice. Briefly, a bicistronic retroviral Bcr-Abl vector (Bcr-Abl-IRES-GFP) was used to produce virus to infect progenitor bone marrow (BM) cells collected from mice previously treated with 5-FU. After 3 rounds of infection, 200,000 GFP positive progenitor BM cells were transplanted into lethally irradiated hosts. Two weeks post-BMT, peripheral blood samples were analyzed by FACS analysis to establish the percentage of GFP positive cells present in the recipient mice. The 32 mice included in the study had 10-20% of GFP positive cells. 14 days post-BMT, the mice were stratified into 4 groups of 8 animate each and received a two week treatment with Vehicle, compound A at 80 mg/kg po qd, AMN107 75 mg/kg po qd or the combination between Compound A and AMN107. During the entire study period the mice were followed for any sign of leukemia development, such as hunched position, lost of body weight or inability of grooming. The animate were sacrificed when they reached any of the previously described signs.

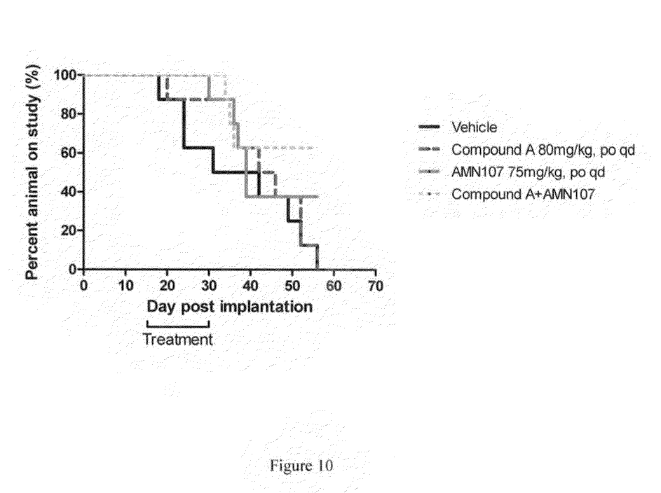

As shown in FIG. 10, all the vehicle and compound A treated animals were sacrificed between 18 to 56 days post-BMT, in the AMN107 treated group 5 animals were sacrificed to date and only 3 mice were sacrificed in the combination group. This suggests an advantage in the combination of these compounds for the treatment of CML.

We claim:

1. A combination comprising a first agent that is a Smoothened inhibitor and a second agent that is a BCR-ABL inhibitor,
wherein the first agent is
a compound of Formula I: 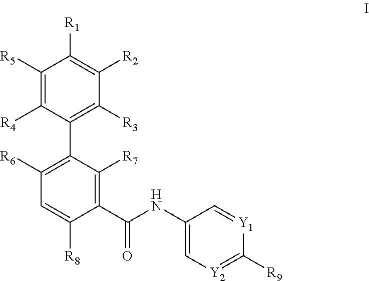
in which
Y₁ and R₂ are independently selected from N and CR₁₀; wherein R₁₀ is selected from hydrogen, halo, C₁₋₆alkyl, halosubstituted-C₁₋₆alkyl, C₁₋₆alkoxy, halosubstituted-C₁₋₆alkoxy and —OXNR₁₀ₐR_{10b}; wherein R₁₀ₐ and R_{10b} are independently selected from hydrogen and C₁₋₆alkyl;
R₁ is selected from cyano, halo, C₁₋₆alkyl, halosubstituted-C₁₋₆alkyl, C₁₋₆alkoxy, halosubstituted-C₁₋₆alkoxy, C₆₋₁₀aryl, dimethyl-amino, C₁₋₆alkyl-sulfanyl and C₃₋₈heterocycloalkyl optionally substituted with up to 2 C₁₋₆alkyl radicals;
R₂ and R₅ are independently selected from hydrogen, cyano, halo, C₁₋₆alkyl, halosubstituted-C₁₋₆alkyl, C₁₋₆alkoxy, halosubstituted-C₁₋₆alkoxy and dimethylamino;
R₃ and R₄ are independently selected from hydrogen, halo, cyano, C₁₋₆alkyl, halosubstituted-C₁₋₆alkyl, C₁₋₆-alkoxy and halosubstituted-C₁₋₆alkoxy; or either R₁ and R₂ or R₁ and R₅ together with the phenyl to which they are both attached form C₅₋₁₀heteroaryl;
R₆ and R₇ are independently selected from hydrogen, C₁₋₆alkyl, halosubstituted-C₁₋₆alkyl, C₁₋₆alkoxy and halosubstituted-C₁₋₆alkoxy; with the proviso that R₆ and R₇ are not both hydrogen;
R₈ is selected from hydrogen, halo, C₁₋₆alkyl, halosubstituted-C₁₋₆alkyl, C₁₋₆alkoxy and halosubstituted-C₁₋₆alkoxy;
R₉ is selected from —S(O)₂R₁₁, —C(O)R₁₁, —OR₁₁, NR₁₂ₐR_{12b} and —R₁₁; wherein R₁₁ is selected from aryl, heteroaryl, cycloalkyl and heterocycloalkyl; R₁₂ₐ and R_{12b} are independently selected from C₁₋₆alkyl and hydroxy-substituted-C₁₋₆alkyl;
wherein said aryl, heteroaryl, cycloalkyl and heterocycloalkyl of R₉ can be optionally substituted with 1 to 3 radicals independently selected from C₁₋₆alkyl, halosubstituted-C₁₋₆alkyl, C₁₋₆alkoxy, halosubstituted-C₁₋₆alkoxy, C₆₋₁₀aryl-C₀₋₄alkyl, C₅₋₁₀heteroaryl-C₀₋₄alkyl, C₃₋₁₂cycloalkyl and C₃₋₈heterocycloalkyl;
wherein said aryl-alkyl substituent of R₉ is optionally substituted with 1 to 3 radicals independently selected from halo, C₁₋₆alkyl, halosubstituted-C₁₋₆alkyl, C₁₋₆alkoxy, halosubstituted-C₁₋₆alkoxy and methyl-piperazinyl; or a pharmaceutically acceptable salt thereof or
a compounds of the formula (II): 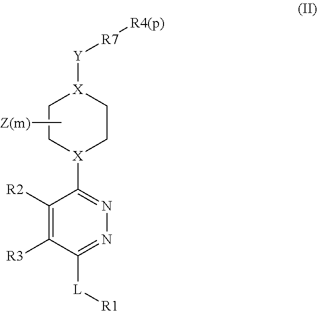
and pharmaceutically acceptable salts thereof, wherein
R1 is a C₆₋₁₄ aryl group, or a 5-14 membered heteroaryl group which may be unsubstituted or substituted;
R2 and R3 are independently C₁₋₈alkyl, C₁₋₈alkylOH, or R2 and R3 form a fused C₃₋₁₄ cycloalkyl group;
L is a bond, C₁₋₈ alkylene, —C(O)O—, —C(O)NR9—, —C₁₋₈alkylOH—, —C₁₋₈ haloalkyl, —C(O)—, —NH— or —O—;
X and W are independently N or CR5, and at least one of X or W is N;
R7 is a C₆₋₁₄ aryl group, a 5-14 membered heteroaryl group, or a 3-14 membered cycloheteroalkyl group;
R4 is C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ alkoxy, halo, NR6R8, C(O)OR6, C(O)NR6R8, C₁₋₈haloalkyl, formyl, carbalkoxy, C₁₋₈alkylOH, C(O)R6, SO₂R6, C(O)NHC₁₋₈alkylR6, NR6R8, SO₂NR6R8, OCF₃, NHC(O)R6, CH₂OC(O)NR6R8, CH₂NR6R8, NHC(O)OR6, NHC(O)NR6R8, CH₂NHSO₂R6, CH₂NHC(O)OR6, OC(O)R6, or NHC(O)R6, which may be substituted or unsubstituted;
Z is C₁₋₈ alkyl, CN, OH, or halogen;
m and p are independently 0-3;
Y is a bond, C₁₋₈ alkylene, —C(O)—, —C(O)O—, —CH(OH)—, or —C(O)NR10;
R5 is H, halogen, CN, lower alkyl, OH, OCH₃ or OCF₃;
Wherein R1 may be substituted by one or more of C₁₋₈ alkyl, a C₆₋₁₄ aryl group, C₁₋₈ haloalkyl, C₁₋₈ alkoxy, halo, NH₂, CN, OCF₃, OH, C(O)NR6R8, C(O)R6, NR6R8, NHC(O)R6, SO₂R6, SO₂NR6R8;
R9 and R10 are independently C₁₋₈ alkyl or H;
R6 and R8 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈haloalkyl, C₁₋₈ alkylOH, C₁₋₈alkoxy, or two R6 on one atom can form a heteroatom containing ring; and
wherein R4, R6, and R8 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH₃, —OCF₃, —OH, —NH₂ or a salt thereof.

2. The combination of claim 1, wherein said first agent is 2-methyl-4′-trifluoromethoxy-biphenyl-3-carboxylic acid [6-(cis, 2,6-dimethyl-morpholin-4-yl)-pyridin-3-yl]-amide or a pharmaceutically acceptable salt thereof.

3. The combination of claim 1, wherein said first agent is 2-[(R)-4-(6-benzyl-4,5-dimethyl-pyridazin-3yl)-2-methyl-3,4,5,6-tetrahydro-2H-[1,2′]bipyrazinyl-5′-yl]-propan-2-ol or a pharmaceutically acceptable salt thereof.

4. The combination of claim 1, wherein said second agent is an ABL inhibitor, and ABL/Scr inhibitor, an Aurora kinase inhibitor, or a non-ATP competitive inhibitor of BCR-ABL.

5. The combination of claim 1, wherein said second agent is selected from the group consisting of nilotinib (AMN107), imatinib (STI571), 2,6,9-trisubstituted purine analogs (e.g., AP23464), AZD-0530, bosutinib (SKI-606), CPG070603, pyrido[2,3-d]pyrimidine compounds (e.g., dasatinib (BMS-354825)), PDI66326, PDI73955, PDI80970), ON012380, 3-substituted benzamide derivatives (e.g., INNO-406), MK-0457 (VX-680), PHA-739358, retaspimycin hydrochloride (IP1-504) and GNF-2.

6. The combination of claim 5, wherein the second agent is nilotinib.

7. The combination of claim 6, wherein the first agent is 2-methyl-4′-trifluoromethoxy-biphenyl-3-carboxylic acid [6-(cis-2,6-dimethyl-morpholin-4-yl)-pyridin-3-yl]-amide or a pharmaceutically acceptable salt thereof.

8. The combination of claim 6, wherein the first agent is 2-[(R)-4-(6-benzyl-4,5-dimethyl-pyridazin-3-yl)-2-methyl-3,4,5,6-tetrahydro-2H-[1,2′]bipyrazinyl-5′-yl]-propan-2-ol or a pharmaceutically acceptable salt thereof.

9. A pharmaceutical composition comprising a first agent that is a Smoothened inhibitor and a second agent that is a BCR-ABL inhibitors,
wherein the first agent is
a compound of Formula I; 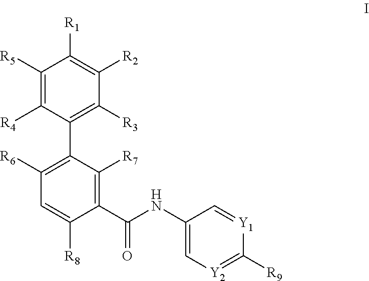
in which
Y₁ and Y₂ are independently selected from N and CR₁₀; wherein R₁₀ is selected from hydrogen, halo, C₁₋₆alkyl, halosubstituted-C₁₋₆alkyl, C₁₋₆alkoxy, halosubstituted-C₁₋₆alkoxy and —OXNR₁₀ₐR_{10b}; wherein R₁₀ₐ and R_{10b} are independently selected from hydrogen and C₁₋₆alkyl;
R₁ is selected from cyano, halo, C₁₋₆alkyl, halosubstituted-C₁₋₆alkyl, C₁₋₆alkoxy, halosubstituted-C₁₋₆alkoxy, C₆₋₁₀aryl, dimethyl-amino, C₁₋₆alkyl-sulfanyl and C₃₋₈heterocycloalkyl optionally substituted with up to 2 C₁₋₆alkyl radicals;
R₂ and R₅ are independently selected from hydrogen, halo, cyano, C₁₋₆alkyl, halosubstituted-C₁₋₆alkyl, C₁₋₆alkoxy, halosubstituted-C₁₋₆alkoxy and dimethylamino;
R₃ and R₄ are independently selected from hydrogen, halo, cyano, C₁₋₆alkyl, halosubstituted-C₁₋₆alkyl, C₁₋₆alkoxy and halosubstituted-C₁₋₆alkoxy; or either R₁ and R₂ or R₁ and R₅ together with the phenyl to which they are both attached form C₅₋₁₀heteroaryl;
R₆ and R₇ are independently selected from hydrogen, C₁₋₆alkyl, halosubstituted-C₁₋₆alkyl, C₁₋₆alkoxy and halosubstituted-C₁₋₆alkoxy; with the proviso that R₆ and R₇ are not both hydrogen;
R₈ is selected from hydrogen, halo, C₁₋₆alkyl, halosubstituted-C₁₋₆alkyl, C₁₋₆alkoxy and halosubstituted-C₁₋₆alkoxy;
R₉ is selected from —S(O)₂R₁₁, —C(O)R₁₁, —OR₁₁, —NR₁₂ₐR_{12b} and —R₁₁; wherein R₁₁ is selected from aryl, heteroaryl, cycloalkyl and heterocycloalkyl; R₁₂ₐ and R_{12b} are independently selected from C₁₋₆alkyl and hydroxy-substituted-C₁₋₆alkyl;
wherein said aryl, heteroaryl, cycloalkyl and heterocycloalkyl of R₉ can be optionally substituted with 1 to 3 radicals independently selected from C₁₋₆alkyl, halosubstituted-C₁₋₆alkyl, C₁₋₆alkoxy, halosubstituted-C₁₋₆alkoxy, C₆₋₁₀aryl, C₀₋₄alkyl, C₅₋₁₀heteroaryl-C₀₋₄alkyl, C₃₋₁₂cycloalkyl and C₃₋₈heterocycloalkyl;
wherein said aryl-alkyl substituent of R₉ is optionally substituted with 1 to 3 radicals independently selected from halo, C₁₋₆alkyl, halosubstituted-C₁₋₆alkyl, C₁₋₆alkoxy, halosubstituted-C₁₋₆alkoxy and methyl-piperazinyl; or a pharmaceutically acceptable salt thereof or
a compounds of the formula (II): 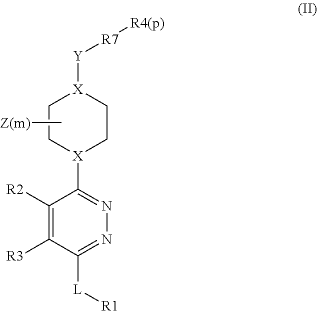
and pharmaceutically acceptable salts thereof, wherein
R1 is a C₆₋₁₄ aryl group, or a 5-14 membered heteroaryl group which may be unsubstituted or substituted;
R2 and R3 are independently C₁₋₈ alkyl, C₁₋₈ alkylOH, or R2 and R3 form a fused C₃₋₁₄ cycloalkyl group;
L is a bond, C₁₋₈ alkylene, —C(O)O—, —C(O)NR9—, —C₁₋₈ alkylOH—, —C₁₋₈ haloalkyl-, —C(O)—, —NH— or —O—;
X and W are independently N or CR5, and at least one of X or W is N;
R7 is a C₆₋₁₄ aryl group, a 5-14 membered heteroaryl group, or a 3-14 membered cycloheteroalkyl group;
R4 is C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈ alkoxy, halo NR6R8, C(O)OR6, C(O)NR6R8, C₁₋₈haloalkyl, formyl, carbalkoxy, C₁₋₈alkylOH, C(O)R6, SO₂R₆, C(O)NHC₁₋₈alkylR6, NR6R8, SO_{NR}6R8, OCF₃, NHC(O)R6, CH₂OC(O)NR6R8, CH₂NR6R8, NHC(O)OR6, NHC(O)NR6R8, CH₂NHSO₂R6, CH₂NHC(O)OR6, OC(O)R6, or NHC(O)R6, which may be substituted or unsubstituted;
Z is C₁₋₈ alkyl, CN, OH, or halogen;
m and p are independently 0-3;
Y is a bond, C₁₋₈ alkylene, —C(O)—, —C(O)O—, —CH(OH)—, or —C(O)NR10;
R5 is H, halogen, CN, lower alkyl, OH, OCH₃ or OCF₃;
Wherein R1 may be substituted by one or more of C₁₋₈ alkyl, a C₆₋₁₄ aryl group, C₁₋₈ haloalkyl, C₁₋₈ alkoxy, halo, NH₂, CN, OCF₃, OH, C(O)NR6R8, C(O)R6, NR6R8, NHC(O)R6, SO₂R6, SO₂NR6R8;
R9 and R10 are independently C₁₋₈ alkyl or H;
R6 and R8 are independently H, C₁₋₈ alkyl, C₂₋₈ alkenyl, C₃₋₁₄ cycloalkyl, a C₆₋₁₄ aryl group, a 5-14 membered heteroaryl group, a 3-14 membered cycloheteroalkyl group, C₁₋₈haloalkyl, C₁₋₈ alkylOH, C₁₋₈alkoxy, or two R6 on one atom can form a heteroatom containing ring; and wherein R4, R6, and R8 can be unsubstituted or substituted by one or more of C₁₋₈ alkyl, C₃₋₁₄ cycloalkyl, a C₆₋₁₄ aryl group, a 5-14 membered heteroaryl group, a 3-14 membered cycloheteroalkyl group, C₁₋₈ alkylOH, OH, oxo, C₁₋₈ haloalkyl, carboxC₁₋₈ alkyl, or SO₂C₁₋₈alkyl, halo, —OCH₃, —OCF₃—, —OH, —NH₂ or a salt thereof.

10. The combination of claim 1, wherein said second agent is selected from the group consisting of nilotinib (AMN107), imatinib (STI571), 2,6,9-trisubstituted purine analogs (e.g., AP23464), AZD-0530, bosutinib (SKI-606), CPG07603, pyrido[2,3-d]pyrimidine compounds (e.g., dasatinib (BMS-354825)), PDI66326, PDI73955PDI80970), ON012380, 3-substituted benzamide derivates (e.g., INNO-406), MK-0457 (VX-680), PHA-739358, retaspimycin hydrochloride (IPI-504) and GNF-2.

11. The composition of claim 10, wherein the second agent is nilotinib.

12. The composition of claim 11, wherein the first agent is 2-methyl-4′-trifluoromethoxy-biphenyl-3-carboxylic acid [6-(cis-2,6-dimethyl-morpholin-4-yl)-pyridin-3-yl]-amide or a pharmaceutically acceptable salt thereof.

13. The composition of claim 11, wherein the first agent is 2-[(R)-4-(6-benzyl-4,5-dimethyl-pyridazin-3-yl)-2-methyl-3,4,5,6-tetrahydro-2H-[1,2′]bipyrazinyl-5′-yl]-propan-2-ol or a pharmaceutically acceptable salt thereof.
